(19) United States
(12) Patent Application Publication
(10) Pub. No.: US 2013/0133645 A1
(43) Pub. Date: May 30, 2013

(54) HEAT AND MOISTURE EXCHANGER

(71) Applicants: Gary C.J. Lee, I-Lan (TW); Yu-Zhi Chen, I-Lan (TW)

(72) Inventors: Gary C.J. Lee, I-Lan (TW); Yu-Zhi Chen, I-Lan (TW)

(21) Appl. No.: 13/547,247

(22) Filed: Jul. 12, 2012

(30) Foreign Application Priority Data
Nov. 25, 2011 (TW) 100143352

(51) Int. Cl. A61M 16/16; A61M 16/04

(52) U.S. Cl. 128/201.13

(57) ABSTRACT

A heat and moisture exchanger of the present invention includes a casing defining a retaining space therein, and an air conditioning unit retained in the retaining space. The casing has a breathing port and an air flow unit that are adapted to communicate fluidly the retaining space, and an air valve unit that has an air hole formed in the casing, and that is adjustable from a closed state to an opened state.

### CROSS-REFERENCE TO RELATED APPLICATION

This application claims priority of Taiwanese Application No. 100143352, filed on Nov. 25, 2011.

### BACKGROUND OF THE INVENTION

1. Field of the Invention

The invention relates to a medical instrument, more particularly to a heat and moisture exchanger (HME).

2. Description of the Related Art

In medical applications, a tracheal tube is generally interconnected fluidly between the trachea and a conventional heat and moisture exchanger (HME) for conveying air therebetween. Exhaled air is conveyed from the trachea to the conventional HME, which serves to condition ambient air using heat and moisture of the exhaled air for provision into the trachea via the tracheal tube, thereby preventing discomfort during inhalation of the filtered air.

However, during use, a filter of the conventional HME may be clogged with secretion from the trachea, which may hinder provision of the ambient air into the trachea and may thus lead to suffocation if clogging of the filter is not eliminated timely (e.g., medical staffs may be too busy to change the clogged filter immediately).

### SUMMARY OF THE INVENTION

Therefore, the object of the present invention is to provide a heat and moisture exchanger (HME) capable of alleviating the aforesaid drawbacks of the prior art.

Accordingly, a heat and moisture exchanger of the present invention is adapted to be connected to a tracheal tube, and comprises:

a casing defining a retaining space therein, and having
a breathing port that is adapted to communicate fluidly the retaining space and the tracheal tube,
an air flow unit that communicates fluidly the retaining space with external environment, and
an air valve unit that has an air hole formed in the casing, and that is adjustable from a closed state, where fluid communication between the retaining space and the external environment through the air hole is blocked, to an opened state, where fluid communication between the retaining space and the external environment through the air hole is permitted; and

an air conditioning unit retained in the retaining space, and serving to condition air that comes from the external environment through the air flow unit and the air valve unit.

### BRIEF DESCRIPTION OF THE DRAWINGS

Other features and advantages of the present invention will become apparent in the following detailed description of the preferred embodiment with reference to the accompanying drawings, of which:

FIG. 1 is a perspective view of a preferred embodiment of a heat and moisture exchanger (HME) according to the invention;

FIG. 2 is an exploded perspective view of the preferred embodiment, illustrating an assembly relationship with an oxygen tube;

FIG. 3 is a perspective view of a casing and a filter member of the preferred embodiment;

FIG. 4 is an exploded perspective view of the casing of the preferred embodiment;

FIG. 5 is another exploded perspective view of the casing of the preferred embodiment;

FIG. 6 is a front view of the casing of the preferred embodiment, illustrating an air valve unit in a closed state; and

FIG. 7 is a view similar to FIG. 6, but illustrating the air valve unit in an opened state.

### DETAILED DESCRIPTION OF THE PREFERRED EMBODIMENT

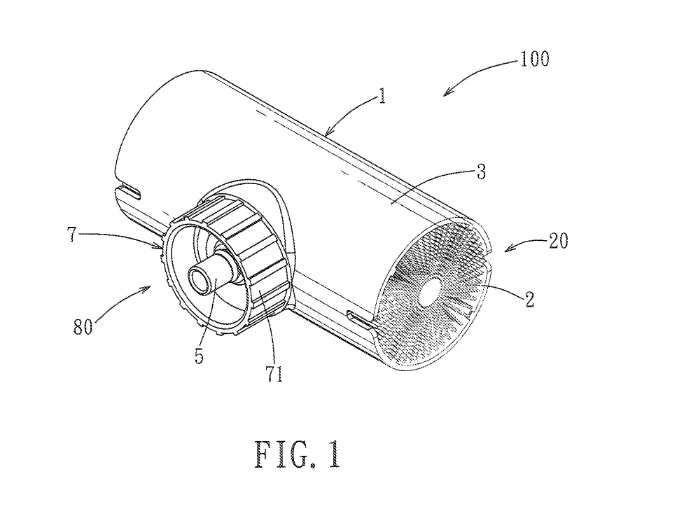

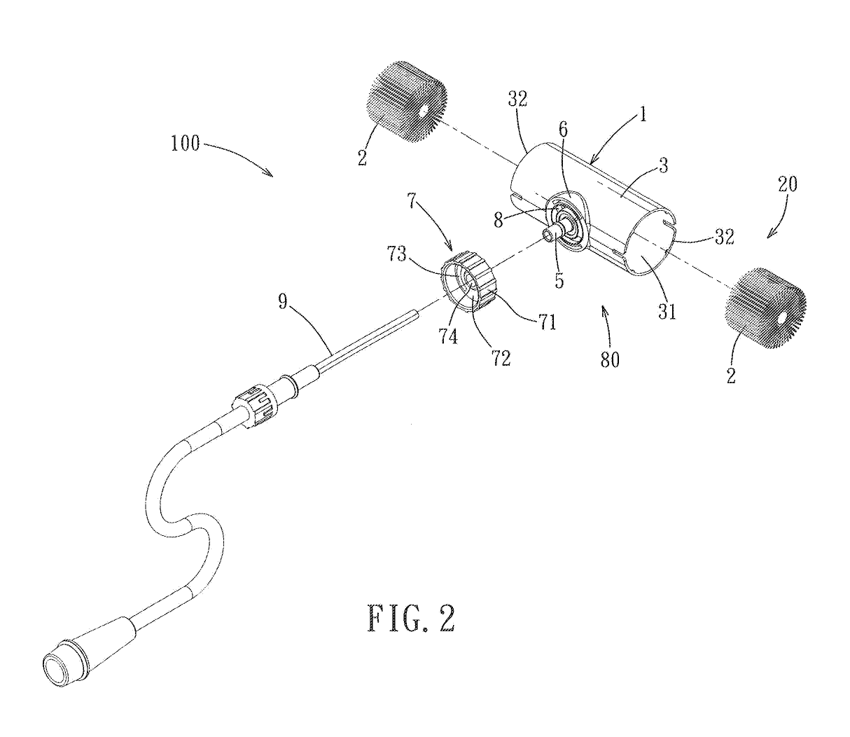

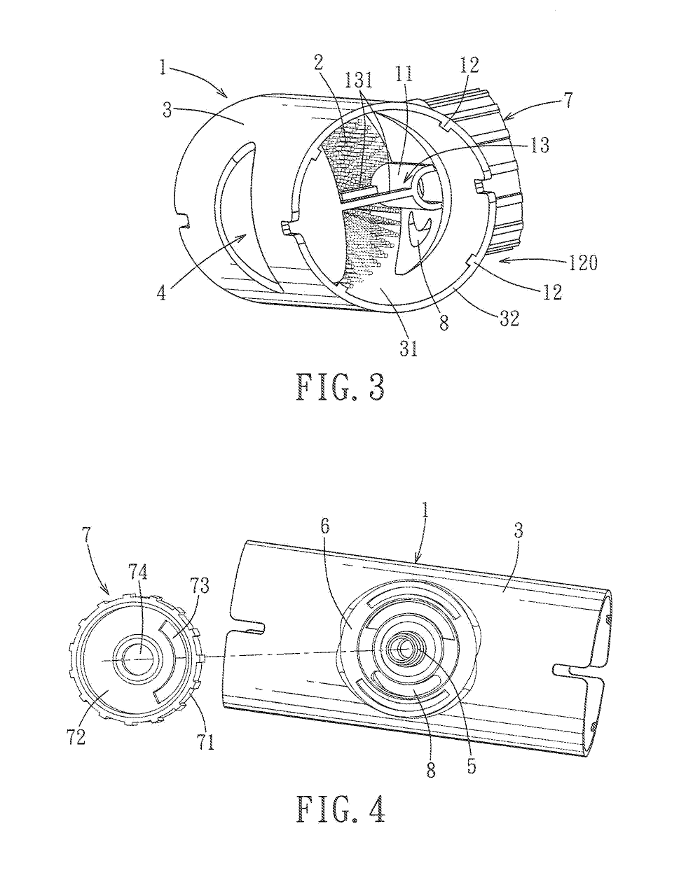

As shown in FIGS. 1, 2 and 3, a preferred embodiment of a heat and moisture exchanger (HME) 100 according to the present invention is adapted to be connected fluidly to a tracheal tube (not shown), and to an oxygen-supply device (not shown) via an oxygen tube 9, and comprises a casing 1 and an air conditioning unit 20.

The casing 1 has a tubular surrounding main wall 3 that defines a retaining space 31 therein, and that has opposite ends in a longitudinal direction. The casing 1 further has an air flow unit that communicates fluidly the retaining space 31 with external environment, and that has a pair of openings 32 formed respectively in the opposite ends of the surrounding main wall 3 and communicating fluidly the retaining space 31. The air conditioning unit 20 includes a pair of filter members 2 retained in the retaining space 31 and spaced apart from each other. Each of the filter members 2 is inserted fittingly in a respective one of the openings 32.

The casing 1 further has a breathing port 4 that is adapted to be connected to the tracheal tube and that communicates fluidly the retaining space 31 and the tracheal tube. In this embodiment, the tracheal tube is inserted air-tightly into the breathing port 9. The air flow unit further has an oxygen inlet portion 5 that is connected to the oxygen tube 9 and that communicates fluidly the retaining space 31 and the oxygen tube 9. In this embodiment, the breathing port 4 and the oxygen inlet portion 5 are formed between the filter members 2 and are formed respectively in diametrically-opposite sides of the surrounding main wall 3.

The casing 1 further has a guiding wall 11, a pair of protrusion units 120, and a limiting wall unit 13. The guiding wall 11 is connected to an inner surface of the surrounding main wall 3 and disposed at a position corresponding to the oxygen inlet portion 5 to guide oxygen that enters into the retaining space 31 through the oxygen inlet portion 5 to flow toward the filter members 2. The protrusion units 120 correspond respectively in position to the openings 32. Each of the protrusion units 120 includes a plurality of protrusions 12 disposed angularly around a respective one of the openings 32, and protrude radially and inwardly from an inner surface of the surrounding main wall 3 for holding a corresponding one of the filter members 2 in the respective one of the openings 32. The limiting wall unit 13 is disposed between the breathing port 4 and the guiding wall 11 for limiting extension of the tracheal tube into the retaining space 31. In this embodiment, the limiting wall unit 13 includes a pair of stepped wall segments 131 adapted for abutting against the tracheal tube when the tracheal tube is inserted into the breathing port 4, thereby preventing over-insertion of the tracheal tube that would otherwise affect adversely the humidification of air in the retaining space 31 and detachment of the tracheal tube from the breathing port 4.

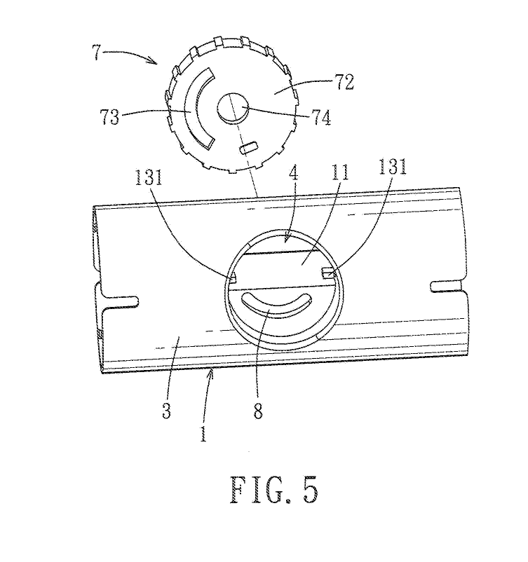

Referring further to FIGS. 4 and 5, the casing 1 further has an air valve unit 80 that has a ring-shaped fitting seat 6 disposed on an outer surface of the surrounding main wall 3 at the diametrically-opposite side of the surrounding main wall 3 with respect to the breathing port 4. The air valve unit 80 further has an air hole 8 formed in the surrounding main wall 3 such that projection of the fitting seat 6 onto the outer surface of the surrounding main wall 3 surrounds projection of th'e air hole 8 onto the outer surface. The air valve unit 80 further has a button unit 7 that has a ring-shaped coupling portion 71 coupled rotatably to the fitting seat 6, and a blocking member 72 connected co-rotatably to the coupling portion 71. The blocking member 72 has the form of a disc, and is configured to extend radially and inwardly from the coupling portion 71, and defines a through hole 74 at center, and an air-communication hole 73 spaced apart from the through hole 74 and not smaller than the air hole 8.

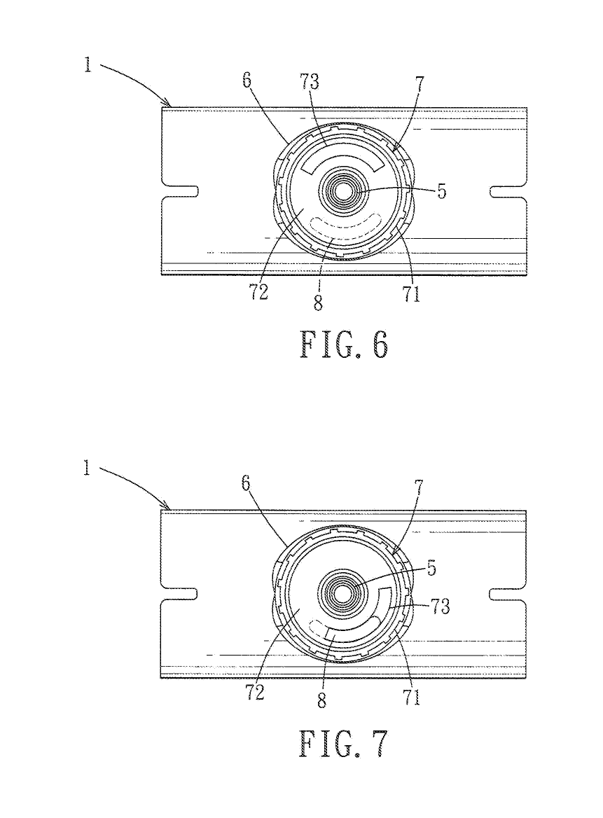

In detail, the oxygen inlet portion 5 is configured to extend through the through hole 74 of the blocking member 72 for being connected to the oxygen tube 9, and the button unit 7 is rotatable about the oxygen inlet portion 5. The air valve unit 80 is adjustable by rotation of the coupling member 71 and the blocking member 72 from a closed state (see FIG. 6), where the air hole 8 is blocked by the blocking member 72 such that fluid communication between the retaining space 31 and the external environment via the air hole 8 is blocked, to an opened state (see FIG. 7), where the air hole 8 is exposed via alignment of the communication hole 73 with the air hole 8 such that fluid communication between the retaining space 31 and the external environment through the air hole 8 is permitted. The presence of the guiding wall 11 keeps the filter members 2 to be spaced apart from the breathing port 4 and the air hole 8 so as to prevent breathing port 4 and the air hole 8 from being blocked by the filter members 2. The air inflow through the air hole 8 can be controlled through adjustment of the percentage of the air hole 8 covered by the blocking member 72 during the rotation of the blocking member 72.

In the beginning of use of the HME 100 of this invention, the air valve unit 80 is in the closed state, and the ambient air is drawn into the retaining space 31 through the openings 31 of the casing 1 by a patient, and filtered, humidified and warmed by the filter members 2. Afterward, the conditioned air is then mixed with oxygen that is drawn into the retaining space 31 through the oxygen inlet portion 5, and eventually flows out of the retaining space 31 through the breathing port 4 for the inhalation of the patient.

After a period of time of use, if the filter members 2 are clogged with secretion while medical staff does not have sufficient time to timely change the filter members 2, the medical staff can operate the air valve unit 80 to the opened state for allowing inflow of extra air into the retaining space 31 therethrough if the filter members 2 are unable to be changed immediately.

As such, the operation of the air valve unit 80 provides extra time for changing the clogged filter members 2 without generating fatal danger to the patient.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A heat and moisture exchanger adapted to be connected to a tracheal tube, comprising:
a casing defining a retaining space therein, and having
a breathing port that is adapted to communicate fluidly said retaining space and the tracheal tube,
an air flow unit that is able to communicate fluidly said retaining space with external environment, and
an air valve unit that has an air hole formed in said casing, and that is adjustable from a closed state, where fluid communication between said retaining space and the external environment via said air hole is blocked, to an opened state, where fluid communication between said retaining space and the external environment via said air hole is permitted; and
an air conditioning unit retained in said retaining space, and serving to condition air that comes from the external environment through said air flow unit and said air valve unit.

2. The heat and moisture exchanger as claimed in claim 1, wherein said air valve unit further has a blocking member that blocks said air hole when said air valve unit is in the closed state.

3. The heat and moisture exchanger as claimed in claim 2, wherein:
said air valve unit further has a ring-shaped fitting seat disposed on an outer surface of said casing, and disposed such that projection of said fitting seat onto said outer surface surrounds projection of said air hole onto said outer surface; and
said blocking member has the form of a disc and is disposed rotatably within said ring-shaped fitting seat.

4. The heat and moisture exchanger as claimed in claim 3, wherein said air valve unit further has a button unit that has a ring-shaped coupling member and said blocking member, said coupling member being coupled rotatably to said fitting seat, said blocking member extending radially and inwardly from said coupling member and defining an air-communication hole that fluidly communicates said retaining space with the external environment via said air hole when said air valve unit is in the opened state;

5. The heat and moisture exchanger as claimed in claim 3, wherein:
said blocking member is further formed with a through hole at center; and
said air flow unit has an oxygen inlet portion that extends from said outer surface of said casing through said through hole and that is adapted to communicate fluidly said retaining space and an oxygen-supply device.

6. The heat and moisture exchanger as claimed in claim 1, wherein:
said casing further has a surrounding main wall defining said retaining space, formed with said breathing port and said air flow unit, said air flow unit having an oxygen inlet portion that is adapted to communicate fluidly said retaining space and an oxygen-supply device;
said air conditioning unit includes a pair of filter members disposed at opposite sides of said oxygen inlet portion; and
said casing further has a guiding wall connected to an inner surface of said surrounding main wall and adapted for guiding oxygen that enters said retaining space through said oxygen port to flow toward said filter members.

7. The heat and moisture exchanger as claimed in claim 6, the tracheal tube being inserted air-tightly into said breathing port, wherein said casing further includes a limiting wall unit disposed between said breathing port and said guiding wall and adapted for limiting extension of the tracheal tube into said retaining space.

8. The heat and moisture exchanger as claimed in claim 6, wherein:
said surrounding main wall of said casing is tubular and has opposite ends in a longitudinal direction;
said air flow unit further has a pair of openings formed respectively in said opposite ends of said surrounding main wall and communicating fluidly said retaining space; and
said filter members are inserted fittingly in said openings, respectively.

9. The heat and moisture exchanger as claimed in claim 8, wherein said casing further has a pair of protrusion units corresponding respectively in position to said openings, each of said protrusion units including a plurality of protrusions that protrude radially and inwardly from an inner surface of said surrounding main wall of said casing for holding a respective one of said filter members in a corresponding one of said openings.
